(19) United States
(12) Patent Application Publication
(10) Pub. No.: US 2012/0101350 A1
(43) Pub. Date: Apr. 26, 2012

(54) Personal Health Monitoring Device

(76) Inventor: David Bychkov, McLean, VA (US)

(21) Appl. No.: 12/910,840

(22) Filed: Oct. 24, 2010

(51) Int. Cl. A61B 5/1455

(52) U.S. Cl. 600/324

(57) ABSTRACT

The present invention provides a system and method for monitoring personal health of the user. The method includes detecting a plurality of physiological parameters. The plurality of physiological parameters are collected and analyzed. Afterwards, the plurality of analyzed physiological parameters are transferred at pre defined time interval to at least one computing device for monitoring the personal health of the user.

### BACKGROUND OF THE INVENTION

1. Field of the Invention

The present invention relates to device for monitoring health of a user. More specifically, the present invention relates to wrist mounted devices for measuring physiological parameters of the user.

2. Description of the Prior Art

Presently, a number of health monitoring devices are available in the market. Most of them detect either one or the other physiological parameters of the user. Some of them are used to measure glucose level of the user while others keep track of the pulse rate of the user. But very few of them allow a single device to measure a combination of the physiological parameters that are important to keep track of the health of the user.

Further, most of the devices available are bulky or are difficult to operate and require the user to visit a doctor or a physician to get regular check up of their health. Therefore, the user is not able to track his or her physiological parameters at all times during the day.

Moreover, all the devices available in the market measure the various physiological parameters of the user independently i.e. there is no correlation between the various physiological parameters. For example, when a user is running the normal range corresponding to the temperature, blood oxygen level and the pulse rate are supposed to be different as compared when the user is at rest. Therefore, high pulse rate during running is normal as compared to high pulse rate while resting.

Also, in case of portable health monitoring devices, very few of them allow the transfer of data corresponding to the physiological parameters to remote computing devices for monitoring by a doctor or a physician.

In the light of the foregoing discussion, there is a need for a portable device that can constantly monitor the important physiological parameters of a user. Further, the device should be able to measure the various physiological parameters in correlation with each other. For example, the normal range of the body temperature, blood oxygen level and the pulse rate should be different depending on the motion information of the user. Also, the device should be able to transfer the readings corresponding to the various physiological parameters to remote computing devices. Additionally, the device should be able to warn the user and/or the doctor in case of an emergency i.e. when the reading corresponding to the one or more physiological parameters of the user falls out of the normal range, the normal range being determined at all times in correspondence with the motion information of the user.

### SUMMARY OF THE INVENTION

An objective of the present invention is to provide a personal health monitoring device.

Another objective of the present invention is to provide a wrist mounted device for measuring plurality of physiological parameters of a user for monitoring personal health of the user.

In accordance with an embodiment of the present invention, the present invention provides a wrist mounted device for personal health monitoring of a user in a non-invasive manner. The wrist mounted device includes an infrared module, a thermistor sensor, a three axis accelerometer, a microcontroller, a wireless networking module and a data storage module. The infrared module further includes one or more infrared (IR) sensors for detecting pulse rate and blood oxygen level of the user. The thermistor sensor detects temperature of the user. The three axis accelerometer monitors motion of the user and configured to generate motion information in response. Further, the microcontroller collects and analyzes a plurality of physiological parameters, the plurality of physiological parameters being at least one of the pulse rate, the blood oxygen level, the temperature and the motion information of the user. Further, the wireless networking module transfers the plurality of physiological parameters to at least one computing device for monitoring personal health of the user. Finally, the data storage module stores the plurality of physiological parameters.

In accordance with another embodiment, the present invention provides a method for personal health monitoring of a user in a non-invasive manner. The method includes detecting a plurality of physiological parameters. The plurality of physiological parameters includes at least one of the pulse rate, the blood oxygen level, the temperature and the motion information of the user. Then, the plurality of physiological parameters are collected. Thereafter, the plurality of physiological parameters is analyzed. Afterwards, the plurality of analyzed physiological parameters are transferred at pre defined time interval to at least one computing device. Finally, the personal health of the user is monitored based on the plurality of physiological parameters.

The device of the present invention possesses a number of advantages. The device of the present invention facilitates monitoring personal health of the user via a wrist mounted device such as a watch. The health of the user can be constantly monitored in a non-invasive manner. Further, the pulse rate, blood oxygen level and the temperature of the user can be constantly monitored in correlation with the motion information of the user for detecting adverse situations. Moreover, the data pertaining to the personal health of the user can be transferred to a remote computing device facilitating monitoring health of the user by a doctor or a physician. Also, the device alerts the user and/or the doctor in case of an emergency.

### BRIEF DESCRIPTION OF THE DRAWINGS

For a more complete understanding of the present invention, the needs satisfied thereby, and the objects, features, and advantages thereof, reference now is made to the following description taken in connection with the accompanying drawings.

FIG. 1 shows a wrist mounted device according to an embodiment of the present invention;

FIG. 2 shows the wrist mounted device according to another embodiment of the present invention;

FIG. 3 shows the wrist mounted device according to yet another embodiment of the present invention;

FIG. 4 shows the wrist mounted device according to still another embodiment of the present invention;

FIG. 5 shows an environment in which various embodiments of the invention can be practiced;

FIG. 6 shows the perspective view of the wrist mounted device according to an embodiment of the device; and,

FIG. 7 shows a flowchart for monitoring personal health of the user according to an embodiment of the present invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION OF THE INVENTION

Before describing in detail the particular device for personal health monitoring of the user in accordance with an embodiment of the present invention, it should be observed that the present invention resides primarily in combinations of system components related to the device of the present invention.

Accordingly, the system components have been represented where appropriate by conventional symbols in the drawings, showing only those specific details that are pertinent to understanding the present invention so as not to obscure the disclosure with details that will be readily apparent to those of ordinary skill in the art having the benefit of the description herein.

In this document, relational terms such as ‘first’ and ‘second’, and the like may be used solely to distinguish one entity or action from another entity or action without necessarily requiring or implying any actual such relationship or order between such entities or actions. The terms ‘comprises’, ‘comprising’, or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by ‘comprises . . . a’ does not, without more constraints, preclude the existence of additional identical elements in the process, method, article, or apparatus that comprises the element.

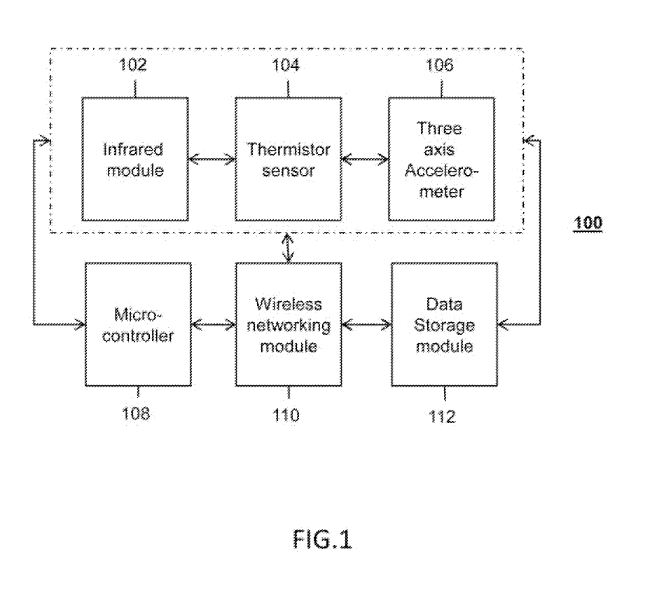

FIG. 1 shows a wrist mounted device 100 according to an embodiment of the present invention. The wrist mounted device 100 includes an infrared module 102, a thermistor sensor 104, a three-axis accelerometer 106, a microcontroller 108, a wireless networking module 110 and a data storage module 112. The infrared module 102 includes one or more infrared sensors. The infrared module 102 detects pulse rate and blood oxygen level of a user.

According to an embodiment of the present invention, the infrared module 102 includes one or more pulse sensors and one or more blood oxygen sensors. The one or more pulse sensors detect the pulse rate of the user. The one or more blood oxygen sensors detect blood oxygen level of the user.

The thermistor sensor 104 detects the temperature of the user. The three-axis accelerometer 106 monitors motion of the user and is configured to generate motion information response. The microcontroller 108 collects and analyzes a plurality of the psychological parameters. The plurality of psychological parameters includes one or more of pulse rate, the blood oxygen level, the body temperature and motion information of the user.

According to an embodiment of the present invention, the microcontroller 108 detects the adverse condition of the user by analyzing temperature, pulse rate and blood oxygen level of the user in correlation with the motion information detected by the three-axis accelerometer 106 i.e. the wrist mounted device 100 alerts the user and/or the doctor in case the reading corresponding to one or more of the temperature of the user, pulse rate and blood oxygen level falls outside the normal range. The normal range varies depending upon the motion information of the user as detected by the three-axis accelerometer 106.

The wireless networking module 110 transfers the plurality of physiological parameters of the user to at least one computing device. Examples of the wireless networking module 110 include, but are not limited to a zigbee module. The wireless networking module 110 transfers the data corresponding to the plurality of physiological parameters by means of a wireless technology. Examples of the wireless technology include, but are not limited to, Bluetooth, Infrared, internet, VPN (Virtual Private Network), mobile operator network and Wi-Fi. Examples of the at least one computing device includes, but are not limited to, a computer, a laptop, a netbook, a PDA and a cellphone.

According to an embodiment of the preset invention, the wireless networking module 108 transfers the plurality of the physiological parameters at pre-defined time-intervals. The pre-defined time intervals can be set by the user or can be preset by the manufacturer of the wrist-mounted device 100.

The data storage module 112 stores the plurality of physiological parameters of the user.

According to an embodiment of the present invention, the data storage module 112 is a memory. The memory can be in-built memory within the wrist-mounted device 100 or can be a removable memory. Examples of the in-build memory include, but are not limited to, Flash memory, ROM and hard disk. Examples of the removable memory include, but are not limited to, Flash memory, hard disk, and memory card.

According to an embodiment of the present invention, the wrist mounted device 100 includes a sub-cover for fixing the one or more infrared sensors. Each of the one or more infrared sensors can be integrated with the sub-cover.

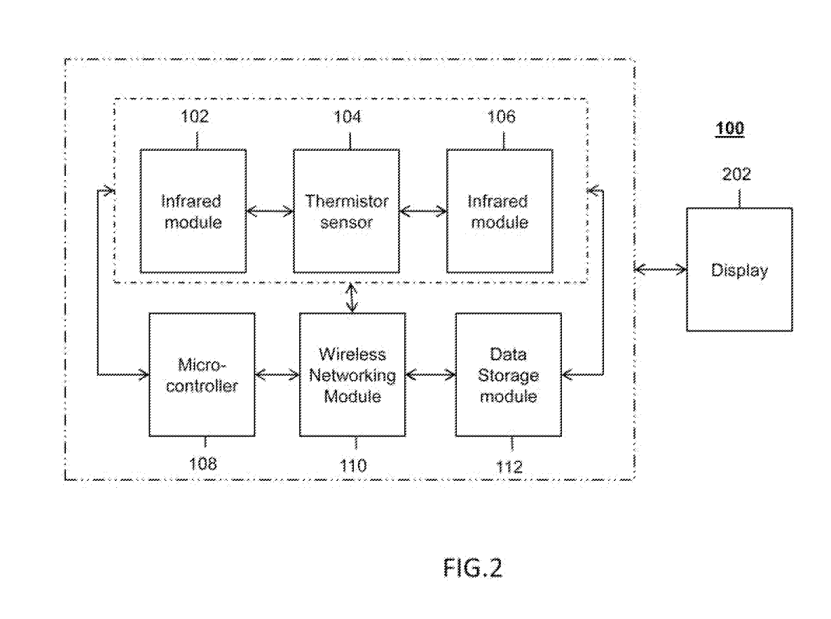

FIG. 2 shows the wrist mounted device 100 according to another embodiment of the present invention. The wrist mounted device 100 includes the infrared module 102, the thermistor sensor 104, the three-axis accelerometer 106, the microcontroller 108, the wireless networking module 110, the data storage module 112 and a display 202. The display 202 is used to display the reading corresponding to the plurality of physiological parameters. Examples of the display 202, include, but are not limited to, a LCD and a touch screen.

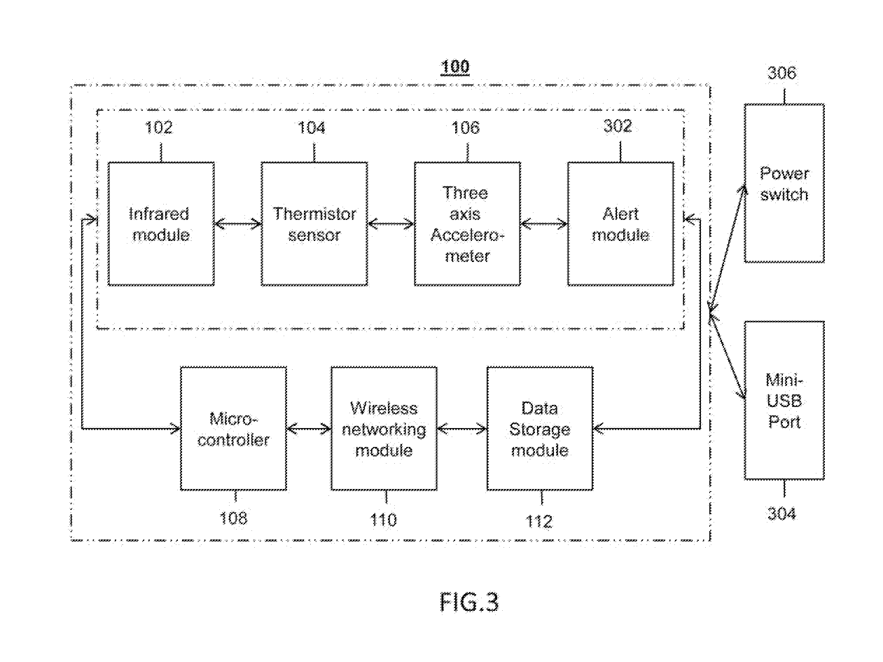

FIG. 3 shows the wrist mounted device 100 according to yet another embodiment of the present invention. The wrist mounted device 100 includes the infrared module 102, the thermistor sensor 104, the three-axis accelerometer 106, the microcontroller 108, the wireless networking module 110, the data storage module 112, an alert module 302, a mini-USB port 304 and a power switch 306. The infrared module 102 includes one or more infrared sensors. The infrared module 102 detects pulse rate and blood oxygen level of a user.

The thermistor sensor 104 detects the temperature of the user. The three-axis accelerometer 106 monitors motion of the user and is configured to generate motion information. The microcontroller 108 collects and analyzes the plurality of the psychological parameters. The plurality of psychological parameters includes one or more of pulse rate, blood oxygen level, body temperature and motion information of the user.

The wireless networking module 110 transferring the plurality of physiological parameters of the user to at least one computing device. Examples of the wireless networking module 110 include, but are not limited to a zigbee module. Examples of the at least one computing device includes, but are not limited to, a computer, laptop, netbook, PDA and a cellphone. The data storage module 112 storing the data corresponding to the plurality of physiological parameters of the user. The alert module 302 alerts the user for one of one of a low battery voltage and an adverse condition of the user.

According to an embodiment of the present invention, the alert module 304 includes a vibrator module and an illumination module. The vibrator module alerts for the adverse condition of the user. The illumination module alerts the user for low battery voltage.

The power switch 304 turns the wrist mounted device 100 either one of on or off. The mini-USB port 306 comprising a mini-USB port for wired communication of the plurality of physiological parameters.

According to an embodiment of the present invention, the mini-USB 306 port can be utilized as a battery charger.

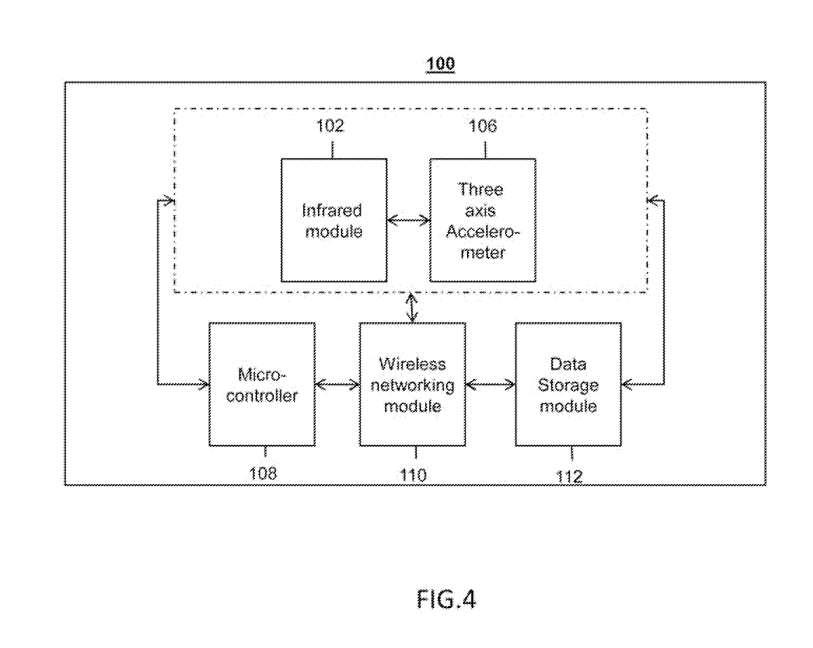

FIG. 4 shows the wrist mounted device 100 according to still another embodiment of the present invention. The wrist mounted device 100 includes the infrared module 102, the three-axis accelerometer 106, the microcontroller 108, the wireless networking module 110 and the data storage module 112. The infrared module 102 includes one or more infrared sensors. The infrared module 102 detects pulse rate and blood oxygen level of a user. The three-axis accelerometer 106 monitors motion of the user and is configured to generate motion information of the user. The microcontroller 108 collects and analyzes the plurality of the psychological parameters. The wireless networking module 110 transferring the plurality of physiological parameters of the user to at least one computing device. Examples of the wireless networking module 110 include, but are not limited to a zigbee module. The data storage module 112 stores the plurality of physiological parameters of the user.

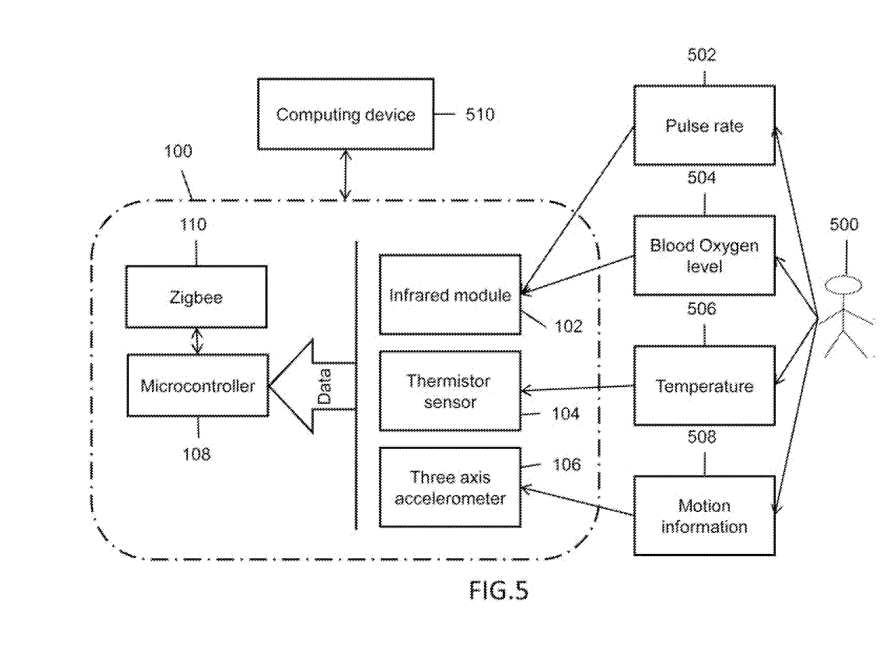

FIG. 5 shows an environment in which various embodiments of the invention can be practiced. The figure shows the wrist mounted device 100 worn by a user 500. The wrist mounted device 100 includes the infrared module 102, the thermistor sensor 104, the three axis accelerometer 106, the microcontroller 108 and the zigbee 110 to detect the plurality of physiological parameters of the user 500. The plurality of physiological parameters includes pulse rate 502, blood oxygen level 504, temperature 506 and motion information 508. The infrared module 102 includes one or more infrared sensors and detects the pulse rate 502 and the blood oxygen level 504. The thermistor sensor 104 detects the temperature 506 of the user 500. The three axis accelerometer 106 detects the motion information 508 of the user 500. The data corresponding to the plurality of physiological parameters is sent to the microcontroller 108. Further, the data is transferred by the zigbee 110 to a computing device 510. The data is sent to the computing device 510 by means of a wireless technology. Examples of the wireless technology include, but are not limited to, Bluetooth, Infrared, internet, VPN (Virtual Private Network), mobile operator network and Wi-Fi. Examples of the computing device 510 includes, but are not limited to, a web-based server, a computer, a laptop, a netbook, a PDA and a cellphone.

According to an embodiment of the present invention, the wrist-mounted device 100 includes a GPS module. The GPS module tracks the location of the user real-time and sends the data to the computing device 510. The user 500 can be located in real-time on occurrence of an emergency via the GPS module. Further, the wrist mounted device 100 can be used to track the health as well as location of a child.

According to another embodiment of the present invention, the wrist-mounted device 100 includes a cellular network module.

According to yet another embodiment of the present invention, the wrist-mounted device 100 includes a camera.

According to still another embodiment of the present invention, the wrist-mounted device 100 includes a microphone.

According to another embodiment of the present invention, the wrist-mounted device 100 includes one or more speakers.

According to yet another embodiment of the present invention, the wrist-mounted device 100 includes a panic button.

According to still another embodiment of the present invention, the wrist-mounted device 100 includes a pre-installed Operating System (OS). Examples of the OS include, but are not limited to, Windows Embedded, Android, Java, Symbian and Windows 7 Mobile.

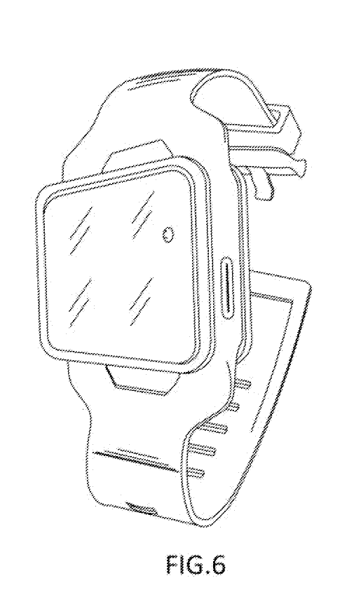

FIG. 6 shows the perspective view of the wrist mounted device 100 according to an embodiment of the device. The wrist mounted device 100 is shown in the shape of a watch. The watch serves the purpose of monitoring the personal health of a user.

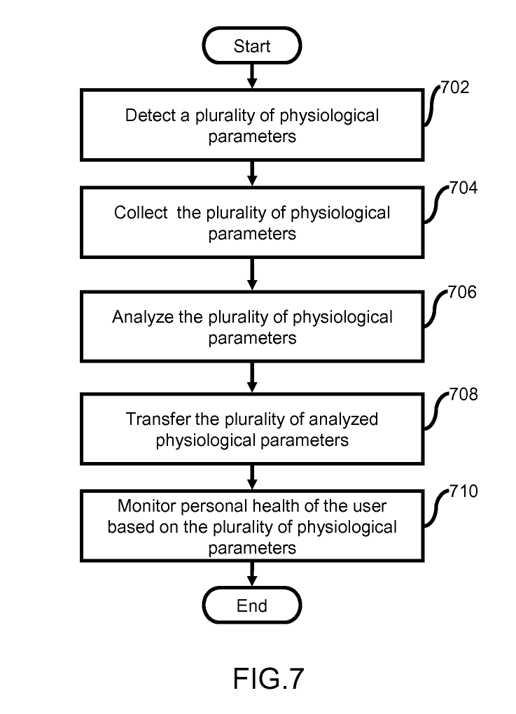

FIG. 7 shows a flowchart for monitoring personal health of the user according to an embodiment of the present invention. At Step 702, a plurality of physiological parameters are detected. The plurality of psychological parameters includes one or more of pulse rate, blood oxygen level, body temperature and motion information of the user.

At step 704, the plurality of physiological parameters is collected. Afterwards, the plurality of physiological parameters are analyzed at step 706.

According to an embodiment of the present invention, adverse condition of the user is detected by analyzing the temperature of the user, pulse rate and blood oxygen level in correlation with the motion information of the user i.e. the user and/or the doctor is alerted in case the reading corresponding to one or more of the temperature of the user, pulse rate and blood oxygen level falls outside the normal range. The normal range varies depending upon the motion information of the user.

Subsequently, the plurality of physiological parameters are transferred at predefined time intervals to at least one computing device at step 708. Thereafter, health of the user is monitored at step 710 based on the plurality of the physiological parameters of the user.

The device of the present invention possesses a number of advantages. The device of the present invention facilitates monitoring personal health of the user via a wrist mounted such as a watch. The health of the user can be constantly monitored. Further, the pulse rate, blood oxygen level and the temperature of the user can be constantly monitored in accordance with the motion information of the user. Moreover, the data pertaining to the personal health of the user can be transferred to a remote computing device facilitating monitoring health of the user by a doctor or a physician, Also, the device alerts the user and/or the doctor in case of any emergency.

While the present invention has been described in connection with preferred embodiments, it will be understood by those skilled in the art that variations and modifications of the preferred embodiments described above may be made without departing from the scope of the invention. Other embodiments will be apparent to those skilled in the art from a consideration of the specification or from a practice of the invention disclosed herein. It is intended that the specification and the described examples are considered exemplary only, with the true scope of the invention indicated by the following claims.

What is claimed is:

1. A wrist mounted device for personal health monitoring of a user in a non-invasive manner, the wrist mounted device comprising:
a) an infrared module, the infra red module including one or more infrared (IR) sensors, the one or more IR sensors for detecting pulse rate and blood oxygen level of the user;
b) a thermistor sensor, the thermistor sensor for detecting temperature of the user;
c) a three axis accelerometer, the three axis accelerometer for monitoring motion of the user and configured to generate motion information in response;
d) a microcontroller, the microcontroller for collecting and analyzing a plurality of physiological parameters, the plurality of physiological parameters including at least one of the pulse rate, the blood oxygen level, the temperature and the motion information of the user;
e) a wireless networking module, the wireless networking module for transferring the plurality of physiological parameters to at least one computing device for monitoring personal health of the user; and
f) a data storage module, the data storage module for storing the plurality of physiological parameters.

2. The wrist mounted device of claim 1, wherein the IR sensor comprises:
a) one or more pulse sensors for detecting pulse rate of the user; and
b) one or more blood oxygen sensors for detecting blood oxygen level of the user.

3. The wrist mounted device of claim 1 further comprising an alert module, the alert module for alerting the user for one of a low battery voltage and an adverse condition of the user.

4. The wrist mounted device of claim 3, wherein the alert module comprises:
a) a vibrator module, the vibrator module alerting for the adverse condition of the user; and
b) an illumination module, the illumination module for alerting the user for low battery voltage.

5. The wrist mounted device of claim 1, wherein the microcontroller detects the adverse condition of the user by analyzing the temperature, pulse rate and blood oxygen level in correlation with the motion information.

6. The wrist mounted device of claim 1 further comprising a mini-USB port for wired communication of the plurality of physiological parameters.

7. The wrist mounted device of claim 6, wherein the mini-USB port is further utilized as a battery charger.

8. The wrist mounted device of claim 1, wherein the zigbee module transfers the plurality of physiological parameters at pre-defined time-intervals.

9. The wrist mounted device of claim 1 further comprising a power switch for turning the wrist mounted device either one of on or off.

10. The wrist mounted device of claim 1 further comprising a sub-cover for fixing the one or more Infra-red (IR) sensors.

11. The wrist mounted device of claim 1 further comprising a display for displaying the reading corresponding to the plurality of physiological parameters of the user.

12. The wrist mounted device of claim 1, wherein the display comprises an LCD screen.

13. A wrist mounted device for personal health monitoring of a user in a non-invasive manner, the wrist mounted device comprising:
a) an infrared module, the infrared module including one or more infrared sensors, the one or more infrared sensors adapted to sense pulse rate and blood oxygen level of the user;
b) a three axis accelerometer, the three axis accelerometer for monitoring motion of the user and configured to generate motion information in response;
c) a microcontroller, the microcontroller for collecting and analyzing a plurality of physiological parameters, the plurality of physiological parameters comprising pulse rate, blood oxygen level, and motion information of the user;
d) a wireless networking module, the wireless networking module for transferring the plurality of physiological parameters to at least one computing device for monitoring the personal health of the user; and
e) a data storage module, the data storage module for storing the plurality of physiological parameters.

14. The wrist mounted device of claim 13, wherein the infrared sensor comprises:
a) one or more pulse sensors for detecting pulse rate of the user; and
b) one or more blood oxygen sensors for detecting blood oxygen level of the user.

15. The wrist mounted device of claim 13 further comprising an alert module, the alert module for alerting the user for one of a low battery voltage and an adverse condition of the user.

16. The wrist mounted device of claim 15, wherein the alert module comprises:
a) a vibrator module, the vibrator module alerting for the adverse condition of the user; and
b) an illumination module, the illumination module for alerting the user for low battery voltage.

17. The wrist mounted device of claim 13, wherein the microcontroller detects the adverse condition of the user by analyzing the temperature, the pulse rate and blood oxygen level in correlation with the motion information.

18. The wrist mounted device of claim 13 further comprising a mini-USB port for wired communication of the plurality of physiological parameters.

19. The wrist mounted device of claim 18, wherein the mini-USB port is further utilized as battery charger of the device.

20. The wrist mounted device of claim 13, wherein the wireless networking module transfers the plurality of physiological parameters at pre-defined time-intervals.

21. The wrist mounted of claim 13 further comprising a thermistor sensor for detecting temperature of the user.

22. The wrist mounted device of claim 13 further comprising a power switch for turning the wrist mounted device either one of on or off.

23. The wrist mounted device of claim 13 further comprising a sub-cover for fixing the one or more Infra-red (IR) sensors.

24. The wrist mounted device of claim 13 further comprising a display for displaying the reading corresponding to the plurality of physiological parameters of the user.

25. The wrist mounted device of claim 24, wherein the display comprises an LCD screen.

26. A method for personal health monitoring of a user in a non-invasive manner, the method comprising:
a) detecting a plurality of physiological parameters, the plurality of physiological parameters including at least one of the pulse rate, the blood oxygen level, the temperature and the motion information of the user;
b) collecting the plurality of physiological parameters;
c) analyzing the plurality of physiological parameters;
d) transferring the plurality of analyzed physiological parameters at pre defined time interval to at least one computing device; and
e) monitoring personal health of the user using the plurality of physiological parameters.

27. The method of claim 26, wherein an alert is sent to the user for low battery voltage and adverse condition of the user.

28. The method of claim 27, wherein an adverse condition of the user is detected by analyzing the temperature, the pulse rate and blood oxygen level in correlation with the motion information of the user.
